(19) United States
(12) Patent Application Publication
(10) Pub. No.: US 2025/0281659 A1
(43) Pub. Date: Sep. 11, 2025

(54) AIR FRESHENER ASSEMBLY FOR A MICROWAVE OVEN APPLIANCE

(71) Applicant: Haier US Appliance Solutions, Inc., Wilmington, DE (US)

(72) Inventor: Bagawathkumar Chellappan, Prospect, KY (US)

(21) Appl. No.: 18/601,478

(22) Filed: Mar. 11, 2024

(51) Int. Cl. A61L 9/12; F24C 15/32; H05B 6/64

(52) U.S. Cl. CPC A61L 9/12; F24C 15/322; H05B 6/6402; A61L 2209/15

(57) ABSTRACT

A microwave oven appliance may define a vertical direction, a lateral direction, and a transverse direction. The microwave oven appliance may include a cabinet that may define a cooking chamber. The microwave oven appliance may further include a door rotatably coupled to the cabinet to provide selective access to the cooking chamber. The microwave oven appliance may also include a ventilation system provided within the cabinet and configured to circulate air. The ventilation system may include an exhaust vent. The microwave oven appliance may further include a freshener assembly attached to the microwave oven appliance downstream from the exhaust vent. The freshener assembly may include a base selectively attached to the cabinet. The base may define a socket. The freshener assembly may further include a holder assembly movably mounted to the socket. The holder assembly may include a moveable control arm configured to receive a scent cartridge.

### FIELD OF THE INVENTION

The present subject matter relates generally to a microwave oven appliance, and more particularly, to an air freshener assembly for a microwave oven appliance.

### BACKGROUND OF THE INVENTION

Microwave oven appliances generally include a cabinet that defines a cooking chamber for receipt of food items for cooking. These appliances typically include one or more heating elements for generating energy to heat the food items during a cooking process. For example, microwave ovens typically include at least one source of electromagnetic radiation in the microwave frequency range, such as a cavity magnetron. Generally, microwave oven appliances include an air handler or fan to circulate air either through the appliance or through an ambient atmosphere around the appliance (e.g., throughout the kitchen). These fans can blow air out from the appliance via an exhaust vent.

Food and other items heated or cooked within the kitchen (e.g., within the cooking chamber or cavity or within an oven) can produce strong odors. These odors may emanate from the appliance and linger in and around the room in which the microwave is situated (such as a kitchen or kitchenette). Users of the appliance and any patrons within the room or space may find the odors to be unpleasant. However, current methods for reducing kitchen odors have several drawbacks. For instance, separate scent releasers plugged into different outlets may reduce odors in places away from the source of the odor, resulting in less than desirable effects.

Accordingly, a microwave oven appliance that obviates one or more of the above-mentioned drawbacks would be beneficial.

### BRIEF DESCRIPTION OF THE INVENTION

Aspects and advantages of the invention will be set forth in part in the following description, or may be obvious from the description, or may be learned through practice of the invention.

In one exemplary aspect of the present disclosure, a microwave oven appliance is provided. The microwave oven appliance may define a vertical direction, a lateral direction, and a transverse direction. The microwave oven appliance may include a cabinet that may define a cooking chamber. The microwave oven appliance may further include a door rotatably coupled to the cabinet to provide selective access to the cooking chamber. The microwave oven appliance may also include a ventilation system provided within the cabinet and configured to circulate air. The ventilation system may include an exhaust vent. The microwave oven appliance may further include a freshener assembly attached to the microwave oven appliance downstream from the exhaust vent. The freshener assembly may include a base selectively attached to the cabinet. The base may define a socket. The freshener assembly may further include a holder assembly movably mounted to the socket. The holder assembly may include a moveable control arm configured to receive a replaceable scent cartridge.

In another exemplary aspect of the present disclosure, a microwave oven appliance is provided. The microwave oven appliance may include a cabinet. The cabinet may define a cooking chamber. The microwave oven appliance may also include a ventilation system provided within the cabinet and configured to circulate air. The ventilation system may include an exhaust vent. The microwave oven appliance may further include a freshener assembly removably attached to the microwave oven appliance at the exhaust vent. The freshener assembly may include a base selectively attached to the cabinet. The base may define a socket and a groove. The freshener assembly may further include a holder assembly movable mounted to the socket and the groove. The holder assembly may include a ball positioned within the socket to form a ball joint. The holder assembly may further include a sleeve positioned within the groove and extended from the ball. The holder assembly may also include a moveable control arm configured to receive a replaceable scent cartridge.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures.

FIG. 1 provides a front view of a microwave oven appliance according to exemplary embodiments of the present disclosure.

FIG. 2 provides a perspective view of the exemplary microwave oven appliance of FIG. 1.

FIG. 3 provides a perspective view of an exemplary freshener assembly according to one or more exemplary embodiments of the present subject matter.

FIG. 4 provides a side view of the exemplary freshener assembly of FIG. 3.

FIG. 5 provides a top view of the exemplary freshener assembly of FIG. 3 with a replaceable scent cartridge removed.

FIG. 6 provides a cross-section view of the exemplary freshener assembly of FIG. 5 taken along line A-A.

Repeat use of reference characters in the present specification and drawings is intended to represent the same or analogous features or elements of the present invention.

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope of the invention. For instance, features illustrated or described as part of one embodiment can be used with another embodiment to yield a still further embodiment. Thus, it is intended that the present invention covers such modifications and variations as come within the scope of the appended claims and their equivalents.

As used herein, the terms “first,” “second,” and “third” may be used interchangeably to distinguish one component from another and are not intended to signify location or importance of the individual components. The terms “includes” and “including” are intended to be inclusive in a manner similar to the term “comprising.” Similarly, the term “or” is generally intended to be inclusive (i.e., “A or B” is intended to mean “A or B or both”). In addition, here and throughout the specification and claims, range limitations may be combined and/or interchanged. Such ranges are identified and include all the sub-ranges contained therein unless context or language indicates otherwise. For example, all ranges disclosed herein are inclusive of the endpoints, and the endpoints are independently combinable with each other. The singular forms “a,” “an,” and “the” include plural references unless the context clearly dictates otherwise.

Approximating language, as used herein throughout the specification and claims, may be applied to modify any quantitative representation that could permissibly vary without resulting in a change in the basic function to which it is related. Accordingly, a value modified by a term or terms, such as “generally,” “about,” “approximately,” and “substantially,” are not to be limited to the precise value specified. In at least some instances, the approximating language may correspond to the precision of an instrument for measuring the value, or the precision of the methods or machines for constructing or manufacturing the components and/or systems. For example, the approximating language may refer to being within a 10 percent margin, i.e., including values within ten percent greater or less than the stated value. In this regard, for example, when used in the context of an angle or direction, such terms include within ten degrees greater or less than the stated angle or direction, e.g., “generally vertical” includes forming an angle of up to ten degrees in any direction, e.g., clockwise or counterclockwise, with the vertical direction V.

The word “exemplary” is used herein to mean “serving as an example, instance, or illustration.” In addition, references to “an embodiment” or “one embodiment” does not necessarily refer to the same embodiment, although it may. Any implementation described herein as “exemplary” or “an embodiment” is not necessarily to be construed as preferred or advantageous over other implementations. Moreover, each example is provided by way of explanation of the invention, not limitation of the invention. In fact, it will be apparent to those skilled in the art that various modifications and variations can be made in the present invention without departing from the scope of the invention. For instance, features illustrated or described as part of one embodiment can be used with another embodiment to yield a still further embodiment. Thus, it is intended that the present invention covers such modifications and variations as come within the scope of the appended claims and their equivalents.

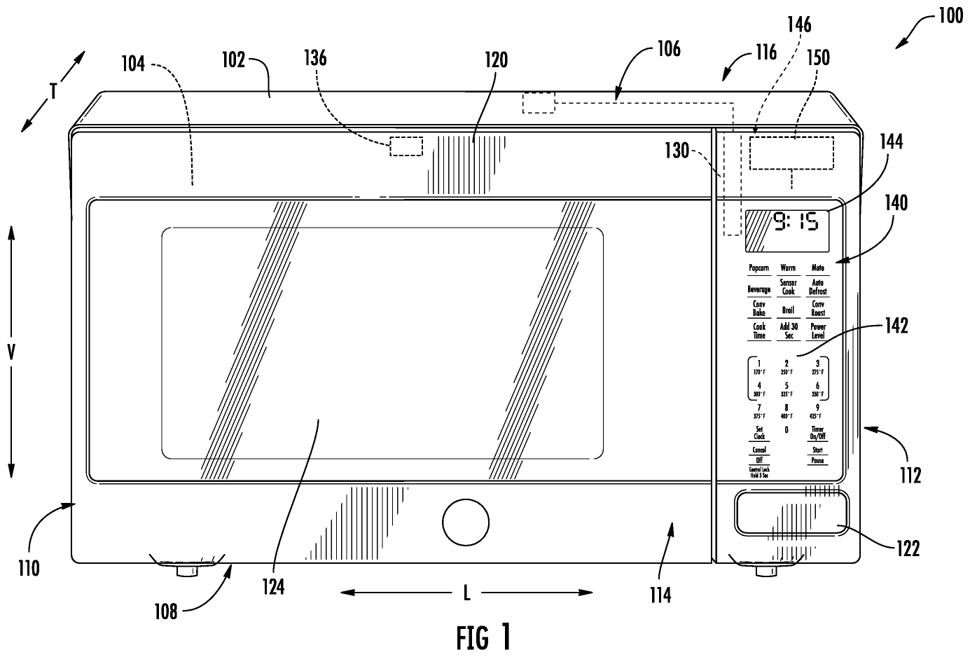

Referring now to the figures, FIG. 1 provides a front view of a microwave oven 100 as may be employed with the present subject matter. Microwave oven 100 includes an insulated cabinet 102 that defines a cooking chamber 104 for receipt of food items for cooking. As will be understood by those skilled in the art, microwave oven 100 is provided by way of example only, and the present subject matter may be used in any suitable microwave oven, such as a countertop microwave oven, an over-the-range microwave oven, etc. Thus, the example embodiment shown in the figures is not intended to limit the present subject matter to any particular cooking chamber configuration or arrangement.

As illustrated, microwave oven 100 generally defines a vertical direction V, a lateral direction L, and a transverse direction T, each of which is mutually perpendicular, such that an orthogonal coordinate system is generally defined. Cabinet 102 of microwave oven 100 extends between a top 106 and a bottom 108 along the vertical direction V, between a first side 110 (left side when viewed from front) and a second side 112 (right side when viewed from front) along the lateral direction L, and between a front 114 and a rear 116 along the transverse direction T.

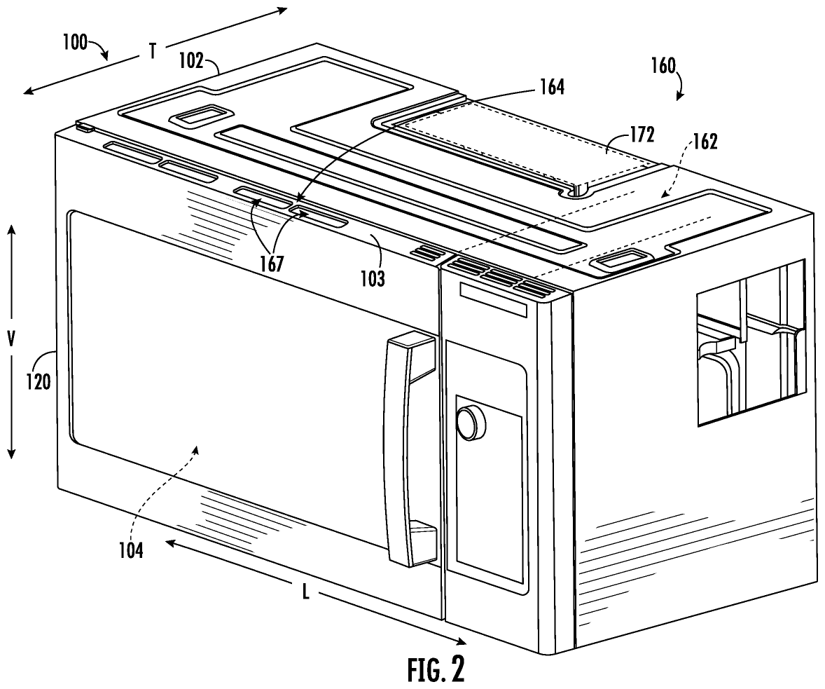

Microwave oven 100 includes a door 120 that is rotatably attached to cabinet 102 in order to permit selective access to cooking chamber 104. A handle (FIG. 2) may be mounted to door 120 to assist a user with opening and closing door 120 in order to access cooking chamber 104. As an example, a user can pull on the handle mounted to door 120 to open or close door 120 and access cooking chamber 104. Alternatively, microwave oven 100 may include a door release button 122 that disengages or otherwise pushes open door 120 when depressed. Glass windowpanes 124 provide for viewing the contents of cooking chamber 104 when door 120 is closed and also assist with insulating cooking chamber 104.

Microwave oven 100 is generally configured to heat articles, e.g., food or beverages, within cooking chamber 104 using electromagnetic radiation. Microwave oven 100 may include various components which operate to produce the electromagnetic radiation, as is generally understood. For example, microwave oven 100 may include a microwave heating assembly 130 which may include a magnetron (such as, for example, a cavity magnetron), a high voltage transformer, a high voltage capacitor and a high voltage diode.

According to exemplary embodiments, microwave oven 100 may further include an inverter power supply that is operably coupled to microwave heating assembly 130 to provide energy from a suitable energy source (such as an electrical outlet) to microwave heating assembly 130, e.g., the magnetron. The magnetron may convert the energy to electromagnetic radiation, specifically microwave radiation. Microwave heating assembly 130 and/or the inverter power supply may include other suitable components, such as a capacitor that generally connects the magnetron and power supply, such as via high voltage diode, to a chassis. Microwave radiation produced by the magnetron may also be transmitted through a waveguide to cooking chamber 104.

The structure and intended function of microwave ovens are generally understood by those of ordinary skill in the art and are not described in further detail herein. According to alternative embodiments, microwave oven may include one or more heating elements, such as electric resistance heating elements, gas burners, other microwave heating elements, halogen heating elements, or suitable combinations thereof, that are positioned within cooking chamber 104 for heating cooking chamber 104 and food items positioned therein.

Microwave oven 100 may include additional features to improve heating uniformity and precision. For example, according to an exemplary embodiment, microwave oven 100 may include a turntable rotatably mounted within cooking chamber 104. The turntable may be selectively rotated during a cooking process to ensure improved temperature uniformity for the object being heated. In addition, microwave oven 100 may include an infrared temperature sensing array 136 that can measure temperatures across the entire bottom of the cooking chamber 104. Temperature sensing array 136 may detect temperatures at various distinct temperature locations, may associate certain locations with the food items being cooked, and may use a subset of the temperature data as feedback for regulating the inverter power supply and microwave heating assembly 130 for improved precision. For example, temperature sensing array 136 may include one or more infrared sensors mounted to a top of cooking chamber 104 for periodically or continuously monitoring a surface temperature of the item being heated.

A user interface panel 140 and a user input device 142 may be positioned on an exterior of the cabinet 102. The user interface panel 140 may represent a general purpose Input/Output (“GPIO”) device or functional block. In some embodiments, the user interface panel 140 may include or be in operative communication with user input device 142, such as one or more of a variety of digital, analog, electrical, mechanical or electro-mechanical input devices including rotary dials, control knobs, push buttons, and touch pads. The user input device 142 is generally positioned proximate to the user interface panel 140, and in some embodiments, the user input device 142 may be positioned on the user interface panel 140. The user interface panel 140 may include a display component 144, such as a digital or analog display device designed to provide operational feedback to a user.

Generally, microwave oven 100 may include a controller 150 in operative communication with the user input device 142. The user interface panel 140 of the microwave oven 100 may be in communication with the controller 150 via, for example, one or more signal lines or shared communication busses, and signals generated in controller 150 operate microwave oven 100 in response to user input via the user input devices 142. Input/Output (“I/O”) signals may be routed between controller 150 and various operational components of microwave oven 100. Operation of microwave oven 100 can be regulated by the controller 150 that is operatively coupled to the user interface panel 140.

Controller 150 is a “processing device” or “controller” and may be embodied as described herein. Controller 150 may include a memory and one or more microprocessors, microcontrollers, application-specific integrated circuits (ASICS), CPUs or the like, such as general or special purpose microprocessors operable to execute programming instructions or micro-control code associated with operation of microwave oven 100, and controller 150 is not restricted necessarily to a single element. The memory may represent random access memory such as DRAM, or read only memory such as ROM, electrically erasable, programmable read only memory (EEPROM), or FLASH. In one embodiment, the processor executes programming instructions stored in memory. The memory may be a separate component from the processor or may be included onboard within the processor. Alternatively, a controller 150 may be constructed without using a microprocessor, e.g., using a combination of discrete analog and/or digital logic circuitry (such as switches, amplifiers, integrators, comparators, flip-flops, AND gates, and the like) to perform control functionality instead of relying upon software.

Referring now to FIG. 2, another embodiment of microwave oven 100 will be described. In this embodiment, like reference numerals will be used to refer to like features. The microwave appliance 100 may include on or more air sources configured to circulate air. For instance, the microwave appliance 100 may include a cooling fan for cooling one or more heating sources (e.g., a magnetron) of the microwave appliance 100. Additionally or alternatively, the microwave oven 100 may include a ventilation system 160 (e.g., as illustrated in FIG. 2). For instance, one or more ducts 162 maybe formed within microwave oven 100 (e.g., within cabinet 102) separate from cooking chamber 104. Ducts 162 may facilitate air movement therethrough. For instance, ducts 162 may be in fluid contact with an ambient atmosphere (e.g., within the room in which microwave oven 100 is located). Thus, ventilation system 160 may include an exhaust vent 164. Exhaust vent 164 may define a plurality of apertures 167 (e.g., through which air is exhausted to the ambient atmosphere of the room). For example, a user may select a recirculation ventilation operation to circulate air through the room (e.g., kitchen). The air may thus be released into the room via exhaust vent 164.

Exhaust vent 164 may be formed in cabinet 102. For instance, exhaust vent 164 may be defined at a top (along the vertical direction V) and front (along the transverse direction T) of cabinet 102. According to some embodiments (e.g., as shown in FIG. 2), exhaust vent 164 is formed in a top panel 103 of cabinet 102. Additionally or alternatively, exhaust vent 164 may be formed in a front panel of cabinet 102. Further still, exhaust vent 164 may be formed in door 120. For instance, exhaust vent 164 may be formed in a top of door 120 (e.g., downward along the vertical direction V). Exhaust vent 164 may include a grate 165 that may define the plurality of apertures 167. Each aperture of the plurality of apertures 167 may have any suitable size. For example, each aperture 167 may be elongated along the lateral direction L. However, additional, or alternative sizes or shapes of apertures may be incorporated, and the disclosure is not limited to the examples provided herein.

Ventilation system 160 may include an air handler or fan 172. Fan 172 may be positioned within microwave oven 100 (e.g., within cabinet 102). For instance, fan 172 may be positioned within ducts 162. Accordingly, fan 172 may selectively urge a flow of air through ducts 162 to be exhausted out of exhaust vent 164. Fan 172 may be an axial fan positioned within cabinet 102. However, it should be appreciated that according to alternative embodiments, fan 172 may be positioned at any other suitable location and may be any other suitable fan type, such as a tangential fan, a centrifugal fan, etc.

In addition, according to an exemplary embodiment, fan 172 is a variable speed fan such that it may rotate at different rotational speeds, thereby generating different air flow rates. In this manner, the amount of air circulated through ducts 162 may be continuously and precisely regulated. Moreover, by pulsing the operation of fan 172 or throttling fan 172 between different rotational speeds, the flow of air through ducts 162 may have a different flow velocity or may generate a different flow pattern. Thus, the air may be forced out of exhaust vent 164 via fan 172.

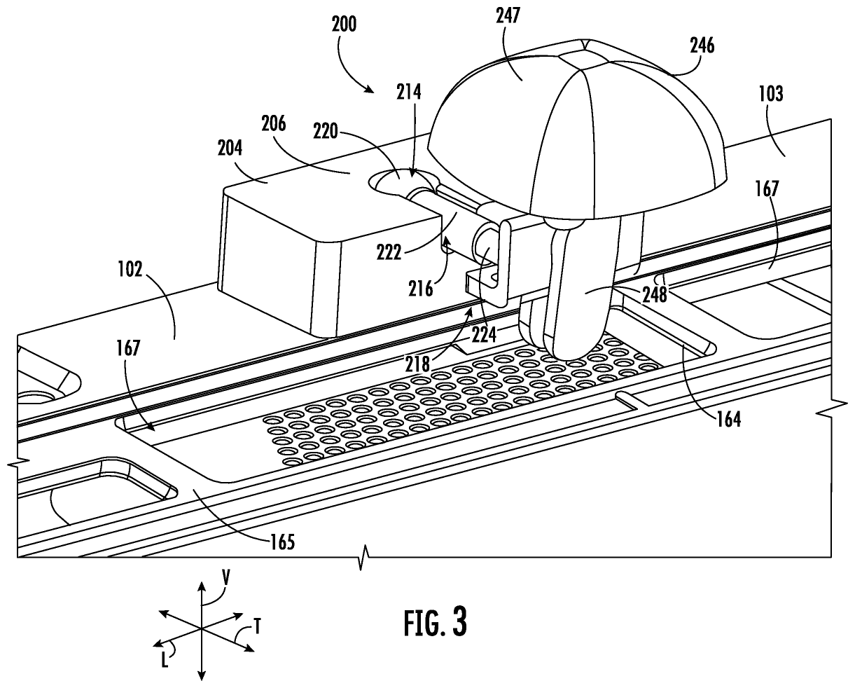

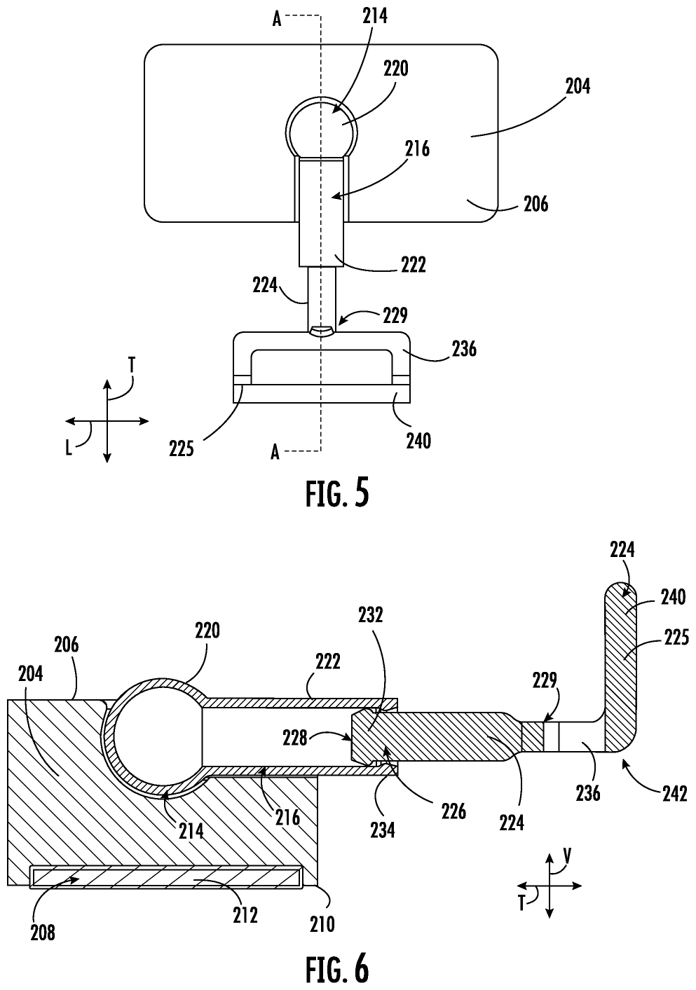

Referring now to FIGS. 3 through 6, a freshener assembly 200 is provided. The freshener assembly 200 may disposed at the microwave oven 100. For instance, the freshener assembly 200 may be selectively attached to microwave oven 100. For instance, freshener assembly 200 may be selectively attached to a portion of the cabinet 102 adjacent to the exhaust vent 164. Particularly, as illustrated in FIG. 3, the freshener assembly 200 may include a base 204 that may be selectively attached or mounted to the top panel 103 of the cabinet 102. The base 204 may define an attachment slot 208 (e.g., FIG. 6) at a bottom surface 210 of the base 204. An attachment assembly 212 may be positioned within the attachment slot 208. The attachment assembly 212 may be utilized to selectively attach the base 204 to the cabinet 102.

The attachment assembly 212 may include any suitable device, apparatus, or component that is capable of attaching or mounting the base 204 to the cabinet 102. For example, the attachment assembly 212 may include a magnet (e.g., permanent magnet). In such embodiments, the attachment assembly 212 (e.g., the magnet) may magnetically attach or mount the base 204 to the cabinet (e.g., as the top panel 103 of the cabinet 102 may be constructed from a magnetic material). As another example, the attachment assembly 212 may include one or more suction cups. In such embodiments, the attachment assembly 212 (e.g., the one or more suction cups) may create a suction fit with the top panel 103 of the cabinet 102. As yet another example, the attachment assembly 212 may include adhesive tape. In such embodiments, the attachment assembly 212 (e.g., the adhesive tape) may couple the base 204 to the top panel 103 of the cabinet 102. As yet another example, the top panel 103 of the cabinet 102 may include an integrated receiver (e.g., a slot or a clip) and the attachment assembly 212 may include a corresponding mount that may be coupled to the receiver. For instance, the attachment assembly 212 may and the top panel 103 of the cabinet 102 may include a snap fit mount assembly 212 for attaching or mounting the base 204 to the cabinet 102.

In some embodiment, the base 204 defines a socket 214 at the top surface 206 of the base 204. Additionally, a horizontal slot or groove 216 may be defined at the top surface 206 of the base 204. The holder assembly 218 may include a ball 220, a sleeve 222, a moveable control arm 224, and a freshener bracket 225. Generally, the ball 220 may be coupled to the base 204 within the socket 214 to form a ball joint.

The socket 214 and the groove 216 may be configured to receive, at least in part, a holder assembly 218. For instance, the holder assembly 218 may include a ball 220, a sleeve 222, a moveable control arm 224, and a freshener bracket 225. The sleeve 222 may be selectively received within the groove 216. In order to permit a limited range of motion in all direction, e.g., via the ball joint, the sleeve 222 may first be lifted out of the groove 216. For instance, the ball 220 may be rotated within the socket 214 such that the sleeve 222 is lifted approximately along the vertical direction V. When the sleeve 222 is freed from the groove 216, the holder assembly 218 may have a limited range of motion in all directions, e.g., via rotation of the ball 220 within the socket 214.

The sleeve 222 may be extended from the ball 220 and may be capable of receiving the moveable control arm 224. The moveable control arm 224 may be slidable or rotatably attached to the holder assembly 218. For instance, the moveable control arm 224 may be telescopically attached within an opening 226 defined by the sleeve 222. The moveable control arm 224 may be capable of extending or retracting between a fully extended position or a fully retracted position. Additionally, the moveable control arm 224 may be capable of extending or retracting in a particular direction with respect to the sleeve 222. For example, when the sleeve 222 is positioned within the groove 216, such as in FIGS. 3 through 6, the moveable control arm 224 may be capable of extending or retracting approximately along the transverse direction T with respect to the sleeve 222.

The moveable control arm 224 may define a first end 228 (e.g., FIG. 6) and a second end 229 spaced apart approximately along the transverse direction T (e.g., when the sleeve is positioned within the groove 216). The moveable control arm 224 may include an annular ring 232 positioned at the first end 228. The annular ring 232 may define a stop point for the moveable control arm 224 (e.g., in the fully extended position). For instance, in the fully extended position, the annular ring 232 may interface with a matching annular groove 234 positioned within the sleeve 222 such that the moveable control arm 224 may not be extended any further.

The freshener bracket 225 may include a first body 236, a second body 238, and an attachment bar 240. When the sleeve 222 is positioned within the groove 216 (e.g., as illustrated in FIGS. 3 through 6), the first body 236 may be extended from the second end 229 of moveable control arm 224 approximately along the transverse direction T. In addition, the second body 238 may be extended from a first end 242 of the first body 236 approximately along the vertical direction V. The attachment bar 240 may be positioned at a proximal end 244 of the second body 238.

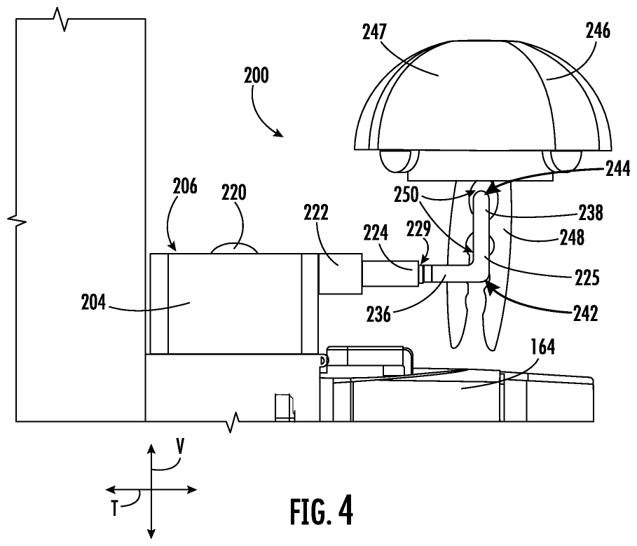

Freshener assembly 200 may be configured to receive a replaceable scent cartridge 246 (e.g., FIGS. 3 and 4). For instance, freshener assembly 200 may provide a space into which the replaceable scent cartridge 246 may be mounted. The replaceable scent cartridge 246 may be attached to the attachment bar 240 of the freshener bracket 225. The replaceable scent cartridge 246 may include a scent pod 247 and a clip 248 extended from the scent pod 247. The scent pod 247 may have scent-releasing capabilities. For example, when airflow from the exhaust vent 164 passes over or through the scent pod 247, a scent held within the scent pod 247 may be released. Additionally or alternatively, the scent pod 247 may contain an odor absorber (e.g., baking soda, activated charcoal, hydrogen peroxide, etc.) to absorb odors produced during heating processes within microwave oven 100. In this regard, the scent pod 247 may advantageously eliminate or mitigate odors that may be emitted from the exhaust vent 164 during operation of the microwave oven 100.

In some embodiments, the clip 248 defines one or more attachment grooves 250. For example, as illustrated in FIG. 4, the attachment grooves 250 may be positioned approximately along the vertical direction V. Each attachment groove 250 may be configured to receive the attachment bar 240. In this regard, each attachment groove 250 defined by the clip 248 may define one or more clip positions for the replaceable scent cartridge 246. The one or more clip positions may each define a distance from the scent pod 247 to the attachment bar 240. Changing the distance from the scent pod 247 to the attachment bar 240 may allow a user to adjust how close the scent pod 247 is to the exhaust vent 164. In this regard, a user may be capable of controlling an amount of airflow that passes through or over the scent pod 252 (e.g., during operation of the microwave oven 100). Therefore, an amount of scent that may be released from the scent pod 247 may advantageously be adjusted.

In some embodiments, the attachment assembly 212 may advantageously facilitate selective attachment of the freshener assembly 200 to the top surface 206 of the cabinet 102. For example, the attachment assembly 212 may allow the freshener assembly 200 to be attached a microwave oven or retrofitted onto an existing microwave oven. Additionally or alternatively, the ball joint formed between the socket 214 and the ball 220 may advantageously allow a user to adjust an angle of the replaceable scent cartridge 246 relative to the exhaust vent 164. For instance, a user may adjust the angle to alter the amount of airflow that may pass into or through the scent pod 2447. In this regard, a user may advantageously be capable of adjusting the amount of scent released from the scent pod 247 during operation of the microwave oven 100. Additionally or alternatively, the moveable control arm 224 may advantageously allow a user to adjust a distance between the replaceable scent cartridge 246 and the cabinet 102. In this regard, a user may be capable of further adjusting the positioning of the replaceable scent cartridge 246, for instance, to accommodate various sized or shaped replaceable scent cartridges 246 or to adjust the amount of scent that may be released from the scent pod 247 during operation of the microwave oven 100.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

What is claimed is:

1. A microwave oven appliance defining a vertical direction, a lateral direction, and a transverse direction, the microwave oven appliance comprising:
a cabinet defining a cooking chamber;
a door rotatably coupled to the cabinet to provide selective access to the cooking chamber;
an air source provided within the cabinet and configured to circulate air, the air source comprising a vent at the exterior of the cabinet; and
a freshener assembly attached to the microwave oven appliance, the freshener assembly comprising:
a base selectively attached to the cabinet, the base defining a socket; and
a holder assembly comprising a moveable control arm configured to receive a scent cartridge,
wherein the holder assembly is movably mounted to the socket to selectively position the scent cartridge downstream of the vent.

2. The microwave oven appliance of claim 1, wherein the holder assembly comprises a ball and a sleeve extended from the ball, and wherein the ball is positioned within the socket to form a ball joint.

3. The microwave oven appliance of claim 2, wherein the holder assembly further comprises a bracket attached to the moveable control arm, and wherein the scent cartridge is disposed at the bracket.

4. The microwave oven appliance of claim 3, wherein the sleeve defines an opening, and wherein the moveable control arm is telescopically attached to the sleeve within the opening.

5. The microwave oven appliance of claim 3, wherein the bracket comprises a first body, a second body, and an attachment bar, wherein the first body is extended from the moveable control arm approximately along the transverse direction, wherein the second body is extended from the first body approximately along the vertical direction, and wherein the attachment bar is positioned at a proximal end of the second body.

6. The microwave oven appliance of claim 5, wherein the scent cartridge comprises a clip coupled to the attachment bar of the bracket.

7. The microwave oven appliance of claim 1, wherein the base defines an attachment slot at a bottom surface thereof, and wherein an attachment assembly is disposed within the attachment slot.

8. The microwave oven appliance of claim 7, wherein the base is selectively attached to the cabinet via the attachment assembly.

9. The microwave oven appliance of claim 8, wherein the attachment assembly includes a magnet, and wherein the magnet is magnetically attached to a top panel of the cabinet.

10. The microwave oven appliance of claim 8, wherein the attachment assembly includes one or more suction cups, and wherein the one or more suction cup create a suction fit between the attachment assembly and a top panel of the cabinet.

11. A microwave oven appliance defining a vertical direction, a lateral direction, and a transverse direction, the microwave oven appliance comprising:
a cabinet, the cabinet defining a cooking chamber;
a ventilation system provided within the cabinet and configured to circulate air, the ventilation system comprising an exhaust vent;
a freshener assembly removably attached to the microwave oven appliance at the exhaust vent, the freshener assembly comprising:
a base selectively attached to the cabinet, the base defining a socket and a groove; and
a holder assembly movable mounted to the socket and the groove, the holder assembly comprising a ball positioned within the socket to form a ball joint, a sleeve positioned within the groove and extended from the ball, and a moveable control arm configured to receive a scent cartridge.

12. The microwave oven appliance of claim 11, wherein the sleeve defines an opening, and wherein the moveable control arm is telescopically attached to the sleeve within the opening.

13. The microwave oven appliance of claim 11, wherein the holder assembly further comprises a bracket attached to the moveable control arm, and wherein the scent cartridge is disposed at the bracket.

14. The microwave oven appliance of claim 13, wherein the bracket comprises a first body, a second body, and an attachment bar, wherein the first body is extended from the moveable control arm approximately along the transverse direction, wherein the second body is extended from the first body approximately along the vertical direction, and wherein the attachment bar is positioned at a top of the second body.

15. The microwave oven appliance of claim 14, wherein the scent cartridge comprises a clip coupled to the attachment bar of the bracket.

16. The microwave oven appliance of claim 11, wherein the base defines an attachment slot at a bottom surface thereof, and wherein an attachment assembly is disposed within the attachment slot.

17. The microwave oven appliance of claim 16, wherein the base is selectively attached to the cabinet via the attachment assembly.

18. The microwave oven appliance of claim 17, wherein the attachment assembly includes a magnet, and wherein the magnet is magnetically attached to a top panel of the cabinet.

19. The microwave oven appliance of claim 17, wherein the attachment assembly is one or more suction cups, and wherein the one or more suction cup create a suction fit between the attachment assembly and a top panel of the cabinet.
